(19) United States
(12) Patent Application Publication
(10) Pub. No.: US 2009/0239233 A1
(43) Pub. Date: Sep. 24, 2009

(54) SINGLE-TUBE, READY-TO-USE ASSAY KITS, AND METHODS USING SAME

(75) Inventors: Michael W. Hunkapiller, San Carlos, CA (US); Dennis A. Gilbert, San Francisco, CA (US); Kenneth J. Livak, San Jose, CA (US); Junko Stevens, Menlo Park, CA (US); Susan K. Eddins, San Diego, CA (US); Michael Lucero, South San Francisco, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 12/408,870

(22) Filed: Mar. 23, 2009

Related U.S. Application Data
(63) Continuation of application No. 10/404,640, filed on Apr. 1, 2003, now abandoned.
Application No. 10/404,640 is a continuation-in-part of application No. 10/335,690, filed on Jan. 2, 2003, now abandoned.
(60) Provisional application No. 60/450,733, filed on Feb. 28, 2003.
(60) Provisional application No. 60/369,127, filed on Apr. 1, 2002.
(60) Provisional application No. 60/369,657, filed on Apr. 3, 2002.
(60) Provisional application No. 60/370,921, filed on Apr. 9, 2002.
(60) Provisional application No. 60/376,171, filed on Apr. 26, 2002.
(60) Provisional application No. 60/380,057, filed on May 6, 2002.
(60) Provisional application No. 60/383,627, filed on May 28, 2002.
(60) Provisional application No. 60/390,708, filed on Jun. 21, 2002.
(60) Provisional application No. 60/394,115, filed on Jul. 5, 2002.
(60) Provisional application No. 60/399,860, filed on Jul. 31, 2002.
(60) Provisional application No. 60/352,039, filed on Jan. 25, 2002.
(60) Provisional application No. 60/352,356, filed on Jan. 28, 2002.

(51) Int. Cl. C12Q 1/68

(52) U.S. Cl. 435/6

(57) ABSTRACT

An assay kit is provided that includes assay reagents stored in a single-tube container, and a data storage medium containing information about the contents of the container. Methods are provided for using the data provided with the kit to direct instruments and/or processes, for example, to control an instrument to perform amplification and/or sequencing reactions.

### CROSS REFERENCE TO RELATED APPLICATIONS

This application is a continuation of prior U.S. Non-Provisional patent application Ser. No. 10/404,640, filed Apr. 1, 2003, which claims the benefit under 35 U.S.C. § 119(e) of prior U.S. Provisional Patent Applications No. 60/450,733, filed Feb. 28, 2003, and U.S. Non-Provisional patent application Ser. No. 10/404,640 is a continuation-in-part of prior U.S. Non-Provisional patent application Ser. No. 10/335,690, filed Jan. 2, 2003, and both of said prior Non-Provisional applications claim the benefit under 35 U.S.C. § 119(e) of prior U.S. Provisional Patent Applications Nos.: 60/352,039, filed Jan. 25, 2002, 60/352,356, filed Jan. 28, 2002, 60/369,127, filed Apr. 1, 2002, 60/369,657, filed Apr. 3, 2002, 60/370,921, filed Apr. 9, 2002, 60/376,171, filed Apr. 26, 2002, 60/380,057, filed May 6, 2002, 60/383,627, filed May 28, 2002, 60/390,708, filed Jun. 21, 2002, 60/394,115, filed Jul. 5, 2002, and 60/399,860, filed Jul. 31, 2002, all of which are incorporated herein in their entireties by reference. Cross reference is made to U.S. Provisional Patent Application No. 60/383,954, filed May 29, 2002, and U.S. Non-Provisional patent application Ser. Nos. 10/335,707 and 10/334,793, all of which are incorporated herein in their entireties by reference.

### BACKGROUND

Polymerase chain reaction (PCR) is a common scientific technique for amplification of polynucleotides and deoxyribonucleic acid (DNA) complexes. PCR requires the use of several assay reagents as well as a target analyte, such as a target polynucleotide. The target analyte can be contained in a sample and amplified during a PCR process. During the course of the reaction, the target analyte can be amplified many times. Currently, the assay reagents must be gathered and mixed, typically immediately prior to subjecting the sample to PCR. The many assay reagents are typically obtained from multiple sources and typically mixed together on a laboratory bench top in a physical location that commonly houses laboratory equipment necessary to perform PCR.

### SUMMARY

According to various embodiments, an assay kit is provided that includes: a container containing assay reagents; and a separate data storage medium that contains data about the assay reagents. The assay reagents can be adapted to perform an allelic discrimination or expression analysis reaction when admixed with at least one target polynucleotide. The other reagents can be, for example, components conventionally used for polymerase chain reactions (PCR), and can include non-reactive components. The container can be sealed and can be packaged with the separate data storage medium in a package, for example, in a box. The container can have a machine-readable label that provides information about the contents of the container.

According to various embodiments, the data stored on the data storage medium can include computer-readable code that can be used to adjust, calibrate, direct, set, run, or otherwise control an apparatus, for example, a scientific or laboratory instrument. According to various embodiments, methods are provided wherein the data is used to cause an apparatus to automatically perform a polymerase chain reaction of a target analyte that is mixed with the assay reagents. Methods are also provided whereby the kit is shipped to a customer.

### BRIEF DESCRIPTION OF THE DRAWINGS

FIG. 1 is a top plan segmented view of a plurality of containers in a shipping and storage tray that can be included in a kit, according to various embodiments;

FIG. 2 is a side view of a container of one of the containers shown in FIG. 1;

FIG. 3 is a compact disk that can be included in a kit according to various embodiments;

FIG. 4 is a flowchart outlining a method of use of a container and a data storage medium according to an embodiment; and

FIG. 5 is a schematic diagram of a quenchable dye that can be part of a mixture of reagents according to various embodiments.

It is intended that the specification and examples be considered as exemplary only. The true scope and spirit of the present teachings includes various embodiments.

### DESCRIPTION OF VARIOUS EMBODIMENTS

According to various embodiments, an assay kit is provided that can include, for example: a container containing assay reagents; and a separate data storage medium that contains data about the assay reagents. The assay reagents can include reagents adapted to perform an allelic discrimination or expression analysis reaction when admixed with at least one target polynucleotide sequence. Reagents can be, for example, reagents used for polymerase chain reaction (PCR) amplification, ligase chain reaction (LCR), oligonucleotide reaction assays (OLA), self-sustaining sequence replication, enzyme kinetic studies, homogeneous ligand binding assays, deoxyribonucleic acid or amino acid sequencing, and/or other chemical or biochemical reaction, and can include non-reactive components. The container can be sealed and can be packaged with the separate data storage medium in a package, for example, in a box. The container can have a machine-readable label that provides information about the contents of the container.

According to various embodiments, the terms “polynucleotide” and “DNA,” as used herein, can include nucleic acid analogs that can be used in addition to or instead of nucleic acids. Examples of nucleic acid analogs include the family of peptide nucleic acids (PNA), wherein the sugar/phosphate backbone of DNA or RNA has been replaced with acyclic, achiral, and neutral polyamide linkages. For example, a probe or primer can have a PNA polymer instead of a DNA polymer. The 2-aminoethylglycine polyamide linkage with nucleobases attached to the linkage through an amide bond has been well-studied as an embodiment of PNA and shown to possess exceptional hybridization specificity and affinity. An example of a PNA is as shown below in a partial structure with a carboxyl-terminal amide:

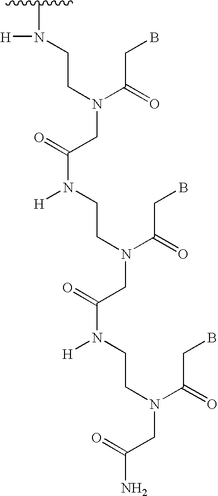

“Nucleobase” as used herein means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases such as, for example, adenine, guanine, cytosine, uracil, thymine, and analogs of the naturally occurring nucleobases, e.g. 7-deazaadenine, 7-deazaguanine, 7-deaza-8-azaguanine, 7-deaza-8-azaadenine, inosine, nebularine, nitropyrrole, nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-azapurine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG”, and ethenoadenine.

“Nucleoside” as used herein refers to a compound consisting of a nucleobase linked to the C-1′ carbon of a sugar, such as, for example, ribose, arabinose, xylose, and pyranose, in the natural β or the α anomeric configuration. The sugar can be substituted or unsubstituted. Substituted ribose sugars can include, but are not limited to, those riboses having one or more of the carbon atoms, for example, the 2′-carbon atom, substituted with one or more of the same or different Cl, F, —R, —OR, —NR₂ or halogen groups, where each R is independently H, C₁-C₆ alkyl or C₅-C₁₄ aryl. Ribose examples can include ribose, 2′-deoxyribose, 2′,3′-dideoxyribose, 2′-haloribose, 2′-fluororibose, 2′-chlororibose, and 2′-alkylribose, e.g. 2′-O-methyl, 4′-α-anomeric nucleotides, 1′-α-anomeric nucleotides, 2′-4′- and 3′-4′-linked and other “locked” or “LNA”, bicyclic sugar modifications. Exemplary LNA sugar analogs within a polynucleotide can include the following structures:

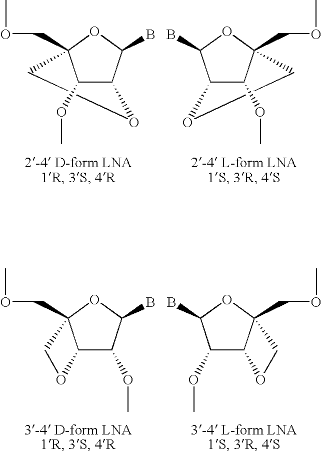

where B is any nucleobase.

Sugars can have modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides can have the natural D configurational isomer (D-form) or the L configurational isomer (L-form). When the nucleobase is a purine, e.g. adenine or guanine, the ribose sugar is attached to the N⁹-position of the nucleobase. When the nucleobase is a pyrimidine, e.g. cytosine, uracil, or thymine, the pentose sugar is attached to the N¹-position of the nucleobase.

“Nucleotide” as used herein refers to a phosphate ester of a nucleoside and can be in the form of a monomer unit or within a nucleic acid. “Nucleotide 5′-triphosphate” as used herein refers to a nucleotide with a triphosphate ester group at the 5′ position, and can be denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates.

As used herein, the terms “polynucleotide” and “oligonucleotide” mean single-stranded and double-stranded polymers of, for example, nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides can have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of internucleotide, nucleobase and sugar analogs. For example, a polynucleotide or oligonucleotide can be a PNA polymer. Polynucleotides can range in size from a few monomeric units, e.g. 5-40 when they are more commonly frequently referred to in the art as oligonucleotides, to several thousands of monomeric nucleotide units. Unless otherwise denoted, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

“Internucleotide analog” as used herein means a phosphate ester analog or a non-phosphate analog of a polynucleotide. Phosphate ester analogs can include: (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Non-phosphate analogs can include compounds wherein the sugar/phosphate moieties are replaced by an amide linkage, such as a 2-aminoethylglycine unit, commonly referred to as PNA.

“Heterozygous” as used herein means both members of a pair of alleles of a gene are present in a sample obtained from a single source, wherein a gene can have two alleles due to, for example, the fusion of two dissimilar gametes with respect to the gene.

“Heterozygous assay” as used herein means an assay adapted to identify the allelic state of a gene having one or both members of a pair of alleles.

“Homozygous” as used herein means one member of a pair of alleles is present in a sample obtained from a single source, wherein a gene can have one allele due to, for example, the fusion of two identical gametes with respect to the gene.

“Homozygous assay” as used herein means an assay adapted to identify only one of two possible allelic states of a gene having one or both members of a pair of alleles.

As used herein, a “sequence detection system” or “SDS” means a laboratory instrument that can perform scientific processes or calculations related to deoxyribonucleic acid sequencing, such as, for example, a polymerase chain reaction, a sequencing reaction, electrophoretic separation, fluorescence detection, and/or basecalling.

A “workflow,” as used herein, is a series of steps that can be performed or processed on automatic laboratory equipment, such as, for example, a liquid handling robot.

“Workflow methods,” as used herein, can include a set of instructions that is followed by the automated laboratory equipment to perform a workflow.

As used herein, the terms “customer” and “user” can be interchangeable.

According to various embodiments, the container can contain all assay reagents and components necessary to conduct PCR, with the exception of a target polynucleotide, also referred to as a target nucleic acid sequence or a target DNA. The target polynucleotide can be provided by a user and mixed with the assay reagents or the target polynucleotide can be provided to the user in a second container to be mixed with the assay reagents. For example, the kit can include a container that contains a target polynucleotide and a container that does not contain a target polynucleotide.

According to various embodiments, the container can be a tube, vial, jar, capsule, ampule, or like vessel. The tube can have a removable cap and/or a replaceable cap. The cap can maintain the container sealed such that the container is water-tight and air-tight. The container can be hermetically sealed. The container can be open at a first end and closed at a second end. According to various embodiments, the first end can be tapered, for example, along the length of the container. According to various embodiments, the container can hold a mixture including assay reagents and have a volume of at least about 5 μL. The container can have a volume of about 10 μL or less. The maximum volume of the container can be about 25 μL or less, and according to other embodiments, the volume can be greater than about 25 μL.

According to various embodiments, standardized assay designs are provided for custom assays and/or stock assays, including either universal concentration or uniform thermal cycling parameters, or both, allowing results to be more easily compared with and/or transferred to other researchers and labs. Also, in some embodiments, assays are formulated in a single-tube 20× mix format that is convenient and easy to use, requiring no preparation or clean-up and providing faster time to results.

According to various embodiments, the assay reagents in the container can contain a volume of assay reagents for more than one respective assay. For example, the assay reagents in the container can be divided and transferred into five respective reaction wells, for example, to conduct five identical and/or different assays. For another example, the assay reagents in the container can be removed from the container and aliquoted into ten respective reaction wells. According to various embodiments, the container can contain a sufficient volume of assay reagents to complete at least 1, at least 5, at least 10, or at least 25 assays.

According to various embodiments, the container can have a label that provides information about the contents of the container. For embodiments including a cap, the label can be secured to the container, or to the cap of the container, if desired. The label can comprise a barcode and can comprise a 2-dimensional barcode that can be provided on the container. In addition to or instead of a barcode, the label can include a serial number, a lot number, a date, and/or other identifying or descriptive indicia. The label can identify the reporter dye or dyes in the container. The label can provide sequence information regarding polynucleotide or peptide reagents provided in the container. The label can contain information about the target polynucleotide sequence, including a common or scientific name or gene name for the target polynucleotide or sequence information about a target analyte. The assay kit can be packaged in, for example, a box. Packaging such as a carton or box can instead or additionally be labeled with identifying and/or descriptive indicia and/or coating as described above.

In addition to a human-readable label, for example, an English-language label with the assay name can be located on each tube. In some embodiments, a 2-D barcode can be laser-etched on the bottom of each assay tube and a 1-D barcode can be laser-etched on each 96-tube rack of assays, thereby making the assay tubes and racks machine-identifiable so that the assays are compatible with automation for high throughput applications.

According to various embodiments, the container can contain at least one probe reactive with a target polynucleotide, wherein the probe can include a polynucleotide, a marker compound, for example, a marker dye, a quenchable dye, or a fluorescent reporter dye, a non-fluorescent quencher, a minor groove binder, or a combination thereof.

The probe can include a reporter dye such as VIC or 6-FAM linked to the 5′ end of the polynucleotide. VIC and 6-FAM dye-labeled probes are available from Applied Biosystems, Foster City, Calif. The minor groove binder can increase the melting temperature Tₘ without increasing the length of the polynucleotide. This can result in greater differences in Tₘ values between matched and mismatched probes that therefore enables more accurate allelic discrimination. The probe can include a quencher (e.g., a non-fluorescent quencher) linked to the 3′ end of the polynucleotide. The quencher can inhibit fluorescence that can facilitate greater discrimination of reporter dye fluorescence.

According to various embodiments, the container can contain two different types of probes, wherein the polynucleotide and the reporter dyes differ. For example, the first type of probe can have a first polynucleotide with a VIC reporter dye attached to the 5′ end of the first polynucleotide and the second type of probe can have a second polynucleotide with a 6-FAM reporter dye attached to the 5′ end of the second polynucleotide and the first and second polynucleotides differ by at least one monomeric unit at the same location in the polynucleotide when the polynucleotides are aligned 5′ to 3′. The dye-labeled probes can be adapted to perform a heterozygous assay or a homozygous assay.

The probe can anneal to a complementary sequence between the forward and reverse primer sites. At the time of annealing, the probe is intact and the proximity of the reporter dye to the quencher can result in suppression of fluorescence of the reporter dye. A polymerase can cleave a reporter dye only when the probe has completely, mostly, or substantially hybridized to the target polynucleotide sequence. When the reporter dye is cleaved from the probe, the relative fluorescence of the reporter dye increases. The increase in relative fluorescence can only occur if the amplified target polynucleotide sequence is complementary, mostly complementary, or substantially complementary to the probe. Therefore, the fluorescent signal generated by PCR amplification can indicate which alleles are present in a sample. Mismatches between a probe and a target polynucleotide sequence can reduce efficiency of probe hybridization and/or a polymerase can be more likely to displace a mismatched probe without cleaving it and therefore not produce a fluorescent signal. For example, if one of two possible reporter dyes fluoresce during an assay, then the presence of a homozygous gene is indicated. For further example, if both possible reporter dyes fluoresce during an assay, then the presence of a heterozygous gene is indicated.

According to various embodiments, the container can contain at least one primer, wherein the primer can comprise a sequence that is shorter than the target polynucleotide. The primer can comprise a polynucleotide and/or a minor groove binder. The primer can comprise a sequence that is complimentary to, or mostly complimentary to, the target polynucleotide. For example, the primer can be at least 90% homologous to a corresponding length of the target polynucleotide, at least 80% homologous to a corresponding length of the target polynucleotide, at least 70% homologous to a corresponding length of the target polynucleotide, or at least 50% homologous to a corresponding length of the target polynucleotide.

According to various embodiments, the container can contain a thermostable DNA polymerase, such as, for example, thermus aquaticus (Taq), and at least 4 embodiments of a deoxyribonucleic acid (e.g., adenosine, tyrosine, cytosine, and guanine). The polymerase can be, for example, AMPLITAQ GOLD, available from Applied Biosystems, Foster City, Calif. According to various embodiments, the container can contain components of a fluorogenic 5′ nuclease assay or other assay reagents that utilize 5′ nuclease chemistry, for example, TAQMAN minor groove binder probes, available from Applied Biosystems, Foster City, Calif. Some or all of the above-listed components can be replaced by or used with commercially-available products, for example, buffers or AMPLITAQ GOLD PCR MASTER MIX (Applied Biosystems, Foster City, Calif.).

According to various embodiments, the assay kit can further include a target polynucleotide in the first or another container, for example, as can be used to prepare a positive control. The assay kit can include more than one container and an Assay Information File (AIF) and/or Electronic Data Sheet (EDS) can be provided on the data storage medium and can contain information about the containers. For example, an AIF or EDS can contain information about 96 containers or 384 containers.

According to various embodiments, a multi-well plate can also be provided in the kit and can include, for example, 96 or 384 positions for container placement. A plate can be substantially rectangular with an optional integrated structural feature for plate orientation. A plate can have a plurality of wells. The assay kit can include a plate adapted to hold a plurality of containers or tubes. The plate can be of unitary construction. One or more containers can be integrated into a single plate, and the plate can have a plurality of containers in physical contact with each other. For example, the plate can be of unitary construction and have 96 containers in the form of receptacles. For further example, the plate can be of unitary construction and have 384 containers.

Assays may be delivered with certain sequence information. For example, some sequence context information (e.g., forward primer location in the RefSeq sequence) can denote which exon-exon junction the assay covers so that users can get a sense of where the assay is positioned in the transcript. More information can be provided, as desired.

For example, according to various embodiments, data can have the following columns (non-limiting examples are listed in paratheticalls following the item): customer name (assigned by the supplier); order number (assigned by the supplier, in some configurations, and can correspond to a number on a 1-D bar code on the plate); ship date (date shipped by the supplier); set ID (an assay name created from record information in the requestor's submission file, including record name and target site name from a target site coordinate; if the sequence record submitted contained multiple target sites, the value of the Set ID can be used to determine which site was used to create the assay); set No. (may be used for internal quality control by the supplier); plate ID (assigned by the supplier, can include the order number value, and can appear on the plate rack as the 1-D bar code); vial ID (a 2-D bar code number can be attached to the bottom of each tube; entry in the datasheet may have leading zeros dropped in some configurations); well location (location of assay tube in the plate rack); line item (may be used for internal quality control by the supplier); VIC probe name (may be used for internal quality control by the supplier); VIC probe sequence (5′ to 3′ sequence of the probe labeled with VIC dye; in some configurations, the 3′ non-fluorescent quencher-minor groove binder (NFQ-MGB) may not be listed but is present on the probe); VIC (μM) concentration (probe concentration); line item (may be used for internal quality control by the supplier); 6-FAM probe name (may be used for internal quality control by the supplier); 6-FAM probe sequence (5′ to 3′ sequence of the probe labeled with 6-FAM dye; in some configurations, the 3′ NFQ-MGB may not be listed but is present on the probe); 6-FAM (μM) concentration (probe concentration); line item (may be used for internal quality control by the supplier); forward primer name (may be used for internal quality control by the supplier); forward primer sequence; forward (μM) primer concentration; line item (may be used for internal quality control by the supplier); reverse primer name (may be used for internal quality control by the supplier); reverse primer sequence; reverse (μM) primer concentration; and/or part number (the part number ordered by the requestor).

The shipped worksheet can be provided to enable a user of the assays to determine that the tubes are in the same positions in the plate rack as when the assays were shipped. For example, according to various embodiments, the following columns can appear in the shipped worksheet: position (position in the plate); and/or vial ID (a 2-D bar code number that can be attached to the bottom of the tube; in some configurations, leading zeros are dropped).

According to various embodiments, the assay kit can be shipped to a customer. The data storage medium can be shipped to the customer along with, concurrent to, separately, previously, or subsequently to shipment of the container and the contents of the container. Alternatively, or additionally, data can be transferred electronically to the customer. The data can be sent to the customer by electronic mail. The customer can retrieve or download the information over a computer network, such as, for example, the Internet, a Wide Area Network, a Local Area Network, or a Virtual Private Network. The customer can retrieve the data using a file transfer protocol or by a hypertext transfer protocol. The protocol can be secured using, for example, 128 bit encryption.

According to various embodiments, the manufactured assays are shipped as homogeneous assays in a single tube format. For example, in at least some embodiments, a single-tube, ready to use format is provided that is suitable for immediate use on an ABI PRISM® Sequence Detection System (SDS) instrument for one or more applications.

Data stored on the data storage medium can include information about a variety of items, for example, a stock number, an assay ID number, a plate number, a well location, a gene symbol or name, a category ID or name, a group ID or name, a chromosome number, a cytogenetic band identification, an NCBI gene reference, an NCBI SNP reference, a minor allele frequency, a minor allele frequency of a particular population, an SNP type, a context sequence, a reporter dye identification, barcode information, or a combination thereof. The context sequence can include, for example, up to 20 bases, more than 20 bases, more than 30 bases, or more than 40 bases. The data stored on the data storage medium can include information about some of the previously-mentioned items, all of the previously-mentioned items, or none of the previously-mentioned items. Furthermore, the information can include more information than that information listed above and/or other identifying or descriptive indicia.

According to various embodiments, the data storage medium can be separate from the container or can be affixed to the container. The data storage medium can be a label attached to the container, for example, with a pressure sensitive adhesive or other glue. The container or cap can serve as the data storage medium and the data can be printed, etched, inscribed, or otherwise encoded on a surface of the container or cap. The data storage medium can include an optically detectable code and/or the data storage medium can be a 2-dimensional barcode.

According to various embodiments, the data can be stored on the data storage medium in electronic format. The data storage medium can be a compact disk (CD). The information can be contained in an Assay Information File (AIF) and/or the Assay Information File can be in the form of an ascii-compatible text file. The data can be contained in an Electronic Data Sheet (EDS) and/or the EDS can be in the form of an ascii-compatible text file. The EDS can contain information that links container identification information, such as, for example, the information contained on a 2-dimensional barcode on the container, to assay identification information. The link can be to assay information contained in, for example, an Assay Information File. The AIF, the EDS, or other computer-readable data or code contained or stored on the data storage medium can be adapted to control an apparatus such as a scientific or laboratory instrument, for example, a liquid handling robot, a thermal cycler, or sequence detection system.

According to various embodiments, the data storage medium can, in addition or in the alternative, contain executable code. The executable code can be in the form of stand-alone software, updates to stand-alone software, or modules to third-party software. The software can be adapted to run on an operating system that controls an SDS instrument, such as, for example, UNIX. The software can include computer code written in assembly language or machine language. The software can be transferred to the SDS instrument by computer and can be saved onto a storage device in the SDS instrument, loaded into a memory device of the SDS instrument, incorporated into previously-loaded software on the SDS instrument, or can be overwritten onto previously-loaded software on the SDS instrument. The storage device can be a hard drive or optical drive. The memory device can be random access memory (RAM) or an erasable, programmable read only memory (EPROM) chip. The software can be provided to a customer on a data storage medium or can be transferred to the customer over a computer network.

According to various embodiments, an Assay Information File (AIF) or Electronic Data Sheet (EDS) can be provided with an assay or assays. The AIF and/or EDS can be, in some embodiments, electronic files or data electronically stored on a data storage medium. The files or data can contain, for example, information on one or more assays, information on one or more polynucleotide sequences, an alphanumeric sequence representing a polynucleotide sequence, or the like. Alternatively, or in addition, a print copy or a printout of the AIF, EDS, and/or information in the AIF and/or EDS can be provided.

According to various embodiments, a printed copy of the AIF and/or EDS can also be provided and can contain information about each assay. This information may include, among other things, the position of each assay in the plate rack. Some embodiments provide, either in place of, or in addition to the printed copy of the AIF and/or EDS, a CD-ROM with one or more data files recorded thereon. The data may include any or all of the following files, and may include other files as well: an electronic assay workbook, including data sheet(s) and shipped worksheet(s); an electronically readable and/or printable copy of instructions for SNP assay protocol for ordering by design; an electronically readable and/or printable copy of protocols for submitting requests; and/or an electronically readable copy of a product insert.

According to various embodiments, a data sheet and/or an electronic assay workbook is provided with custom assays. In some embodiments, an electronic assay workbook is included with each order of up to 92 assays. The workbook file name can include the number on a bar code for easy correlation. The workbook can contain two worksheets, namely, a “data sheet” worksheet and a “shipped” worksheet. The workbook can be a spreadsheet file, such as a MICROSOFT EXCEL spreadsheet software file, that may contain macros and/or be password protected. Cells of the workbook can be copied and pasted into a new worksheet and modified in the new worksheet. A printed copy of the data from the electronic file may be included with a shipment of assays ordered, for example, by design. The data can include a correlation of the 2-D barcodes on the tubes to the corresponding assay names and primer and probe specific information.

According to various embodiments, data included with an order can include at least some of the following information: an identification of the assay in each tube; assay names; which target site was used, if the requester submitted a sequence record that included more than one target site; locations of each tube in the assay rack; sequences of the primers and probes; and concentrations (μM) of primers and probes. Other configurations can necessarily include all of this information and may include more information.

According to various embodiments, a computer program, comprised of lines of machine-readable and/or executable computer code, can obtain data contained in the AIF, EDS, or in another data file, and use the data to control a scientific or laboratory instrument. For example, a microcomputer-based software program can be provided that can load computer-readable data from an AIF or EDS. The AIF or EDS can be stored, for example, on a compact disk that can be shipped to a user along with, concurrent to, previous to, or subsequent to shipping and providing the at least one container of the assay kit. According to various embodiments, the software program can configure, direct, or operate an instrument that can perform PCR, sequencing, and/or sequence detection, for example, an Applied Biosystems 7900HT Sequence Detection System (SDS). The software program can control, for example, an instrument, to perform PCR, sequencing, and/or sequence detection, without human intervention. The SDS can directly operate and run without human interaction. The same or a different software program can perform basecalling of detected sequence data.

According to various embodiments, the kit can include a software program stored on the data storage medium or stored separately on a second data storage medium, for example, a CD. The software program can build an internal assay information database from AIF or EDS files and/or generate plate information adapted to control, at least in part, an SDS instrument. The software can deliver the plate information to the SDS. The software program can flag or note problems with the plate information or plate setup data. The software program can generate an activity log file. The software can detect new plate information setup files, new AIF files, new EDS files, or a combination thereof. The software can import data generated by the customer. The software program can add to, modify, or delete data from an AIF or EDS. The software program can control or direct an SDS instrument to perform, for example, PCR, sequencing, sequence detection, or a combination thereof. The software program can control an SDS instrument to perform PCR, sequencing, or sequence detection using a protocol obtained from an AIF or EDS.

According to various embodiments, the software can receive data from an instrument, for example, from a real-time PCR or SDS instrument, wherein the data is generated during the course of, for example, PCR, sequencing, or sequence detection. An SDS data file can be generated by an SDS instrument and can be transmitted to the software program. The SDS data file can contain information generated by an SDS instrument, or information from an AIF or EDS stored on the data storage medium. The SDS data file can contain error codes or error information generated by the SDS instrument as a result of problems with the SDS instrument, the AIF, and/or the EDS. The data file can include error codes related to, for example, failure to detect at least one fluorescent probe or failure of a component of the SDS instrument, such as a heating element. The SDS instrument can send a log file containing, for example, information about AIF or EDS files transmitted from the software program. A detector list can be generated from an AIF or an EDS and saved in a format suitable for input to a detector manager, such as, for example, an ascii-compatible format.

According to various embodiments, the software can save an AIF, EDS, and/or SDS data file, or other file in separate, respective folders on a data storage device, for example, on a microcomputer. The microcomputer can include, for example, a hard drive, an optical drive, or both. The software program can remain in the memory of a microcomputer before, during, or after transmission of a data file to or from the software program. The software program can continuously monitor data on a microcomputer for new or modified AIF, EDS, and/or SDS data files.

According to various embodiments, a manual method can be used by a customer or user of the assays to validate each tube position in the rack plate. The rack plate position and assay name on the tube label can be compared with the values in the well location and set ID columns of the data. This “validation” is different from the validation of assays, in that validation of each tube position in a plate rack is performed by the user, and merely confirms that the tubes are in positions matching the “shipped” worksheet. If the tubes are not in the correction position, they may be rearranged to match the worksheet. The operational quality of the assays contained within the tubes is validated at the supplier's factory.

According to various embodiments, an automated method can be used by a customer to validate each tube position in the rack plate. This method can include scanning the plate and tubes using a 2-D bar code reader, and executing a plate validation spreadsheet macro (for example, a MICROSOFT EXCEL spreadsheet software macro). To scan the plate and tubes, the plate rack can be placed on the 2-D bar code reader in a standard orientation. For example, tube position “A1” is placed in the top left corner of the reader. The 1-D bar code on the plate rack can then be scanned. The bar code reader can be configured, if necessary, to read positions in one column and to read bar codes in a column next to the positions column. Next, the plate rack is scanned and the results are saved to a director that can be accessed from the computer containing the electronic file. In some configurations, the scanning results are saved as a tab-delimited file.

According to various embodiments, to validate, the “shipped” worksheet can be opened in the spreadsheet and, with macros enabled, the validation macro can be run. In some embodiments utilizing MICROSOFT EXCEL spreadsheet software, the validation is performed by opening the electronic workbook, clicking a mouse on a “shipped” tab to view the worksheet containing the validation macro, clicking on the “validate” button to start the plate validation macro, and, when an “import plate scan” dialog box is presented, selecting “browse” to locate the file from the 2-D bar code scan. After “browse” is selected, the file that resulted from the 2-D bar code scan is selected and imported into a new worksheet, which, in some embodiments, is called “received”. The macro can then compare each bar code and its position in the plate rack with the corresponding bar code in the “shipped” worksheet (for example, a value in the “Vial ID” column). The macro then enters the result in a “validation” column in the “shipped” worksheet. According to various embodiments, the results for each entry may either be “OK” (or any entry understood as indicating a match) or “ERROR” (or any other entry understood as indicating a non-match). A “shipment validation” dialog box can then alert that the validation is complete, and the user clicks “OK” to dismiss the dialog box.

Plate validation errors indicate that the tubes are not in the same position as they were shipped by the supplier to the requestor. The user can resolve plate validation errors by rearranging the tubes to match the “shipped” worksheet. The user can then rescan the plate and execute the validation macro again to validate the plate.

According to various embodiments, an assay kit can be provided that includes at least one assay for allelic discrimination or expression analysis of genomic material. An information source can be provided that has at least one member of the group consisting of an electronic data sheet, an assay information file, and at least one printed datasheet and combinations thereof. The assay can be a SNP assay or a gene expression assay. The assay can be provided in a single tube.

According to various embodiments, the assay can comprise at least one probe and two primers. The assay can be a SNP assay comprising one probe for each of two alleles and two primers. According to various embodiments, the probe can have at least one fluorophore and at least one fluorescence quencher. The fluorescence quencher can be non-fluorescent fluorescence quencher. The probe can have at least one minor groove binder. The assay can have PCR reagents or RT-PCR reagents. The assay can have universal master mix, where the universal master mix has at least one salt, a buffer, and a DNA polymerase.

According to various embodiments, the single tube can have a bar code label. The bar code label can be a two-dimensional bar code label. The single tube can have a human-readable assay number. The kit can be comprised of a plurality of assays, each of which is in a single tube, thereby constituting a plurality of tubes. The plurality of tubes can be contained in a rack and the rack can have a bar-code identification. The kit can also have at least one datasheet containing information on the assay. The kit can have at least one machine-readable medium containing information on the assay. The at least one machine-readable medium can be at least one datasheet containing information on the assay. The machine-readable medium can be a compact disk.

According to various embodiments, data provided to a customer about the contents of containers containing assay reagents can be provided in electronic format. The electronic data can be provided, for example, on compact disk (CD). Data from the CD can be loaded into the memory of a first computer that is in electronic communication with a first laboratory instrument. The electronic data can be changed, manipulated, incorporated into other data, or incorporated into a software program. The electronic data can be loaded into a memory of the first computer by a software program. Software operating on the first computer can use, at least in-part, the electronic data to operate, program, or control the first laboratory instrument. The first laboratory instrument can initiate or perform laboratory methods on assay plates, sample plates, and/or reaction plates provided to the user or provided by the user. Before, during, or after the first laboratory method has been completed, the electronic data can be changed or modified by at least one of the first computer or the first laboratory instrument as a result of the first laboratory method.

According to various embodiments, the electronic data can then be transferred to at least a second computer and can be loaded into the memory of the at least a second computer that is in electronic communication with a second laboratory instrument. The electronic data can be changed, manipulated, incorporated into other data, or incorporated into a software program. Software operating on the at least a second computer can use, at least in-part, the electronic data to operate, program, or control the second laboratory instrument. The second laboratory instrument can initiate or perform laboratory methods on assay plates, sample plates, and/or reaction plates provided to the user or provided by the user. Before, during, or after the second laboratory method has been completed, the electronic data can be changed or modified by at least one of the at least a second computer or the second laboratory instrument as a result of the second laboratory method.

According to various embodiments, the first computer can be the same as the second computer. More than two computers can be used. The electronic data can be provided to a user in an analog format and can be converted to a digital format, for example, by scanning a multi-dimensional barcode into a computer.

According to various embodiments, the first laboratory instrument can be, for example, a liquid handling robot. The second laboratory instrument can be, for example, a sequence detection system. The liquid handling robot can be used to prepare samples and/or assays provided to the user or provided for the user, or combinations thereof, for laboratory analyses by the second laboratory instrument. The second laboratory instrument can amplify and sequence the samples using the assay reagents provided to the user.

According to various embodiments, a workflow can have a sample plate workflow configuration and an assay plate workflow configuration. An assay plate workflow configuration can be a workflow designed to process assays kits according to various embodiments. A sample plate workflow configuration can be a workflow designed to process at least one sample by initiating and performing chemical reactions of the sample using assay kits according to various embodiments. A study can be divided into a series of workflows to complete the study.

On a liquid handling robot, for example, a BIOMEK FX robot, available from Beckman Coulter, Inc., Fullerton, Calif., a sample plate workflow and an assay plate workflow can be combined into a single workflow process. Therefore, the workflow, as used herein, can also include several workflows that are performed simultaneously by automatic laboratory equipment. A workflow can also include more than one sample plate and/or more than one assay plate. The more than one assay plate or the more than one sample plate can be processed sequentially, consecutively, or concurrently. The workflow can also include a reaction plate or plural reaction plates where chemical reactions occur between samples and assays according to various embodiments.

According to various embodiments, a study using a robot can be designed by using at least one of the following steps: determining the number of No Template Controls (NTCs) for the study; dividing the study into workflows; determining the sequence of the workflows; determining volumes necessary to create assay plates; determining volumes necessary to create sample plates; creating an electronic assay plate setup file; selecting a method for the robot to follow; transferring an instruction set to the robot; loading and positioning assay plates, sample plates, and/or reaction plates onto the robot; performing a method using the robot; and combinations thereof. According to various embodiments, after performing the methods, the resulting reaction plates containing samples that have reacted with assays can be transferred to another laboratory instrument, such as, for example, a sequence detection system (SDS).

According to various embodiments, for each workflow, one or more assay plate setup files can be created. The assay plate setup file or assay plate setup file template can be supplied to a user on a CD or other data storage medium. An assay plate setup file can include information needed to control or run the robot. The assay plate setup file can also include information necessary to control or run other equipment, such as, for example, an SDS. The information contained in the assay plate setup file can include sample plate identifiers, assay plate identifiers, reaction plate identifiers, assay ID numbers, and sample ID numbers. The information contained in the assay plate setup file can be the same as the information contained in the AIF and/or EDS. Information found in the assay plate setup file can be provided to the user, entered manually by the user, or scanned by the user, such as, for example, by scanning a barcode or multi-dimensional barcode.

According to various embodiments, a workflow method to control the robot can be selected. The workflow method can be created by the user or the workflow method can be created and provided to the user. According to various embodiments, the assay plate setup file can be the same as an AIF, an EDS, or both. An AIF or EDS can be created automatically using information from the assay plate setup file or vice versa. According to various embodiments, assay kits can comprise a data storage medium containing at least one of an assay plate setup file and an assay plate setup file template. The data storage medium can contain more than one assay plate setup file or assay plate setup template. A user can configure and modify the assay setup file or assay plate setup file template. After the assay plate setup file has been loaded and/or utilized on a robot, the file can be exported from a computer controlling the robot for use by an SDS. Alternately, or additionally, a computer controlling an SDS can load some or all of the information from the assay plate setup file into memory for use by software controlling the SDS. The software controlling the SDS can use only the information contained in the assay plate setup file, some of the information contained in the assay plate setup file, or other information not contained in the assay plate setup file.

According to various embodiments, an assay kit is provided that can include assay reagents contained in at least one 96-tube rack, an assay information file (AIF), and electronic data sheet (EDS), and a product protocol. The protocol, the AIF, and EDS can be in electronic format on a CD. The CD can also contain robot workflow method files, for example, BIOMEK FX method files. The AIF and EDS files can be used with similar systems, assay plate setup file templates, and reaction plate setup files. A software program, for example, SDS Plate Utility, available from Applied Biosystems, Foster City, Calif., can be contained on a CD and can be provided with the assay kit.

The workflow method files for the robot can include at least one workflow method. The at least one workflow method can be used with various configurations of a robot or other instrument. The at least one workflow method can have a corresponding assay plate setup file template. An electronic copy of an instruction or instructions can also be contained on the CD.

Studies using assay kits according to various embodiments can be performed. A study can be designed by determining the number of assays, the number of samples, and the number of no template controls (NTCs) per assay per reaction plate. Using the above information, a series of workflows can be created to allow automated lab equipment, such as, for example, a liquid handling robot to perform at least part of the study. Standard sample plates and assay plates with, for example, 96 wells or 384 wells, can be used. Assays and samples can be selected for use in the workflows. An electronic assay plate setup file template can be used to create an assay plate setup file or files by adding information, such as, for example, sample IDs, assay IDs, and reaction plate barcodes.

To begin a workflow, a workflow method can be loaded into the software application controlling the liquid handling robot and position the plate or plates on the deck of the equipment. Assay plate setup files can be used to keep track of assays, samples, and NTCs that are processed by the automatic laboratory equipment. The workflow can then be run and, after the workflow has been performed, the plates can be removed from the equipment. To preserve the processed samples for later use, the plates can be centrifuged and sealed. Following processing by the automated laboratory equipment, the samples can be processed by other equipment, such as, for example, the ABI PRISM 7900HT SDS DNA Sequencer, available from Applied Biosystems, Foster City, Calif.

According to various embodiments, reaction plate setup can be a part of the workflow used to create and measure reaction plates on laboratory instruments, for example, the 7900HT SDS. Reaction plates can be made using assay plates and/or sample plates. The assay plates and sample plates can be made based on the number of samples and assays to be performed. The product of chemical reactions between the samples and assay reagents, contained in the sample tray and assay tray, respectively, can be contained in corresponding wells of the reaction plate.

The number of samples and assays can be determined. The number of assay plates and the number of sample plates can be determined from the number of samples, the number of assays, and/or the number of controls per assay per plate. The number of workflows of each type needed can be calculated from the above numbers. The workflows can be, for example, BIOMEK FX liquid-handling robot workflows. From the number of workflows of each type, the workflows can be sequenced. To perform a study, each workflow can be performed in sequence.

To start a workflow, the assay plate setup file template for that workflow type can be opened and edited to make an assay plate setup file. Alternatively, or additionally, an assay plate setup file can be provided. Sample IDs and assay IDs can be added to the assay plate setup file. The actual assay plates and sample plates for the workflow can be created using standard layouts. The assay and sample plates can be placed on the robot deck as directed by the workflow method file for that workflow. Pipette tips can be loaded into the tip loader, if necessary or desired. Tips can be manually supplied. The appropriate robot workflow method for the workflow can be run. Bulkheads for the reaction plates made on the robot can be obtained and can be imported to the assay plate setup file or setup files produced by the workflow.

According to various embodiments, the completed assay plate setup file or setup files can be sent to a software program adapted to interact or interface with an SDS to create SDS plate document files, such as, for example, an AIF or EDS. The newly-created reaction plate or plates can be sealed and centrifuged. The reaction plate or plates can then be transferred to the SDS for PCR thermal cycling and/or sequencing. The newly-created reaction plates can be transferred to, for example, the ABI 7900HT instrument for further analysis or processing. The instrument can read the corresponding SDS plate document files based on information from the reaction plates, such as, for example, the reaction plate barcodes. The above-described process can be repeated as necessary or desired.

According to various embodiments, a liquid handling robot can have at least a single robotic arm, a multi channel pipettor, for example, a 96-multi channel pipettor with a capacity of up to 20 microliters, a barcode reader, an automatic labware positioner (ALP) and a stacker carousal. The robot can have two robotic arms. The Biomek FX liquid-handling robot can have a grip space and a deck that allows different labware components to be installed and configured. The deck have ALP to hold components. A barcode reader, a type of ALP, can be attached to a deck position, for example, BR1, and can be used to scan barcodes on plates or labware on the deck. A stacker carousel, another type of ALP, can be placed at, for example, position P1, and can allow storage and exchange of plates and tip boxes from the robot deck. Without a carousel, or similar device, tip boxes can be manually placed and removed in batches during execution of methods. A robot arm can hold a pipettor head and can move objects among deck positions.

According to various embodiments, a multi-channel pipettor can be used to allow direct plate-to-plate transfers and additions of reagents using volumes in a range as required by protocols. The pipettor head can use grippers to move labware and supplies around the deck of the robot. A robot can have, for example, single-arm and/or dual-arm configurations. The dual-arm configuration can occupy a column of the deck layout. Workflow methods can be run on either a single-arm and/or dual-arm configured robot.

According to various embodiments, the total number of combined workflows having a sample workflow and a plate workflow can be calculated in order to set up a workflow method to instruct the robot. The number of combined workflows can be minimized by maximizing the numbers of sample plates and assay plates in each workflow. The workflows can be processed such that a first group of sample plates are processed against a first assay prior to processing a second group of sample plates against a second assay. This arrangement can minimize degradation of assay mixtures and swapping of the assay plates. The combined workflows can be arranged for processing by calculating the number of sample plates, calculating the number of sample plate workflow configurations (e.g., number of 4-plate, 2-plate, and 1-plate sample plates), and calculating the number of combined sample plate and assay plate workflows.

According to various embodiments, once the number and type of workflows has been determined, the order in which the workflows will be processed can be determined.

In the formula SWCn=R/Pc, SWCn=number of n-sample plate reagent workflow configurations, R=number of (96-well) sample plates, and Pc=number of sample plates in workflow configuration. The maximum number of four-sample plate workflow configurations can be calculated using the formula SWC4=S/4, where SWC4=number of four-sample-plate workflow configurations and S=number of (96-well) sample plates.

To determine the types of workflows and how many runs of each type of workflow are needed, the following formulas can be used: BWU1=AWC4×SWC4, BWU2=AWC4×SWC2, BWU3=AWC4×SWC1, BWU4=AWC2×SWC4, BWU5=AWC2×SWC2, BWU6=AWC2×SWC1, BWU7=AWC1×SWC4, BWU8=AWC1×SWC2, and BWU9=AWC1×SWC1, where BWUn=number of n-type BWU runs, SWCn=number of n-sample-plate workflow configurations, and AWCn=number of n-assay-plate workflow configurations. For simplicity, only the four-sample-plate/four-assay-plate workflow (BWU1) can be used. According to such embodiments, the number of such workflows can be found by the formula BWU1=AWC4×SWC4.

The total number of workflows in a study can be found by using the formula: Wtotal=BWU1+BWU2+BWU3+BWU4+BWU5+BWU6+BWU7+BWU8+BWU9, where Wtotal=total number of workflows in a study and BWUn=number of n-type BWU runs.

For example, an example study has 1000 samples, 35 different assays, and four NTCs per sample plate. The following calculations can be performed to optimize the combined workflows, or BWUs, for a Biomek FX robot. The number of sample plates needed can be calculated using the formula Ps=S/(W−NTCsp)=1000/(96−4)=10, with 80 left over. The study requires ten full sample plates and one sample plate with 80 samples, a total of 11 sample plates. The number of four-sample-plate workflow configurations can be calculated using the formula SWC4=R/Pc=11/4=2, with 3 sample plates remainder. The study has two four-sample plate workflow configurations. The number of two-sample-plate workflow configurations can be calculated using the formula SWC2=R/Pc=3/2=1, with 1 sample plate remainder. The study has one two-sample-plate workflow configuration and one one-sample-plate workflow configuration (SWC1=1). Since there are 35 assays and one assay is placed in all 96 wells of a single 96-well assay plate, 35 96-well assays plates are needed.

The number of four-assay-plate workflow configurations can be calculated using the formula AWC4=R/Pc=35/4=8, with 3 assay plates left over. The study has eight four-assay-plate workflow configurations. The number of two-assay-plate workflow configurations can be calculated using the formula AWC2=R/Pc=3/2=1, with 1 assay plate left over. The study has one two-assay-plate workflow configurations and one one-assay-plate workflow configuration (AWC1=1).

The numbers and types of BWUs needed in the study can be calculated. The number for each sample-plate/assay-plate workflow can be calculated by the formulas BWU1=AWC4×SWC4=8×2=16, BWU2=AWC4×SWC2=8×1=8, BWU3=AWC4×SWC1=8×1=8, BWU4=AWC2×SWC4=1×2=2, BWU5=AWC2×SWC2=1×1=1, BWU6=AWC2×SWC1=1×1=1, BWU7=AWC1×SWC4=1×2=2, BWU8=AWC1×SWC2=1×1=1, and BWU9=AWC1×SWC1=1×1=1.

The total number of workflows can be calculated using the formula Wtotal=40=BWU1+BWU2+BWU3+BWU4+BWU5+BWU6+BWU7+BWU8+BWU9. Therefore, the study has 40 BWUs.

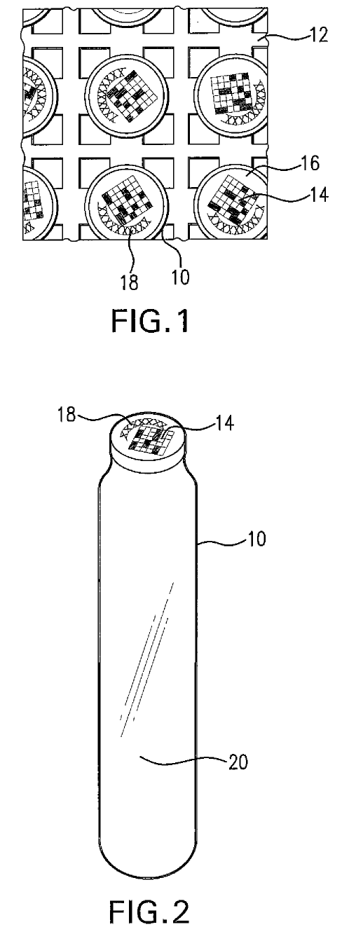

Referring to the drawing figures, FIG. 1 is a plan sectional view of an embodiment of an assay kit having several containers 10 that are held in place for shipping in a tray 12. A 2-dimensional barcode 14 is located on the top side of a container cap 16. The 2-dimensional barcode 14 is adapted to be machine readable. A serial number 18 is located next to the 2-dimensional barcode 14. The serial number 18 is printed in Arabic numerals.

FIG. 2 is a side view of one of the containers 10 shown in FIG. 1. The container 10 has a 2-dimensional barcode 14 and a serial number 18 printed on a cap 16 for the container 10. The cylindrical body 20 of the container 10 is adapted to hold a mixture of assay reagents and is adapted to fit into a tray 12 for shipping and storage.

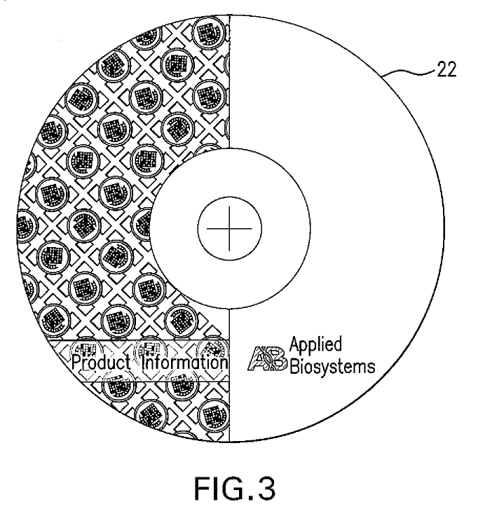

FIG. 3 is a compact disk 22 that can be included in an assay kit according to various embodiments, and can be provided to a user along with a container (not shown in FIG. 3) containing a mixture including assay reagents.

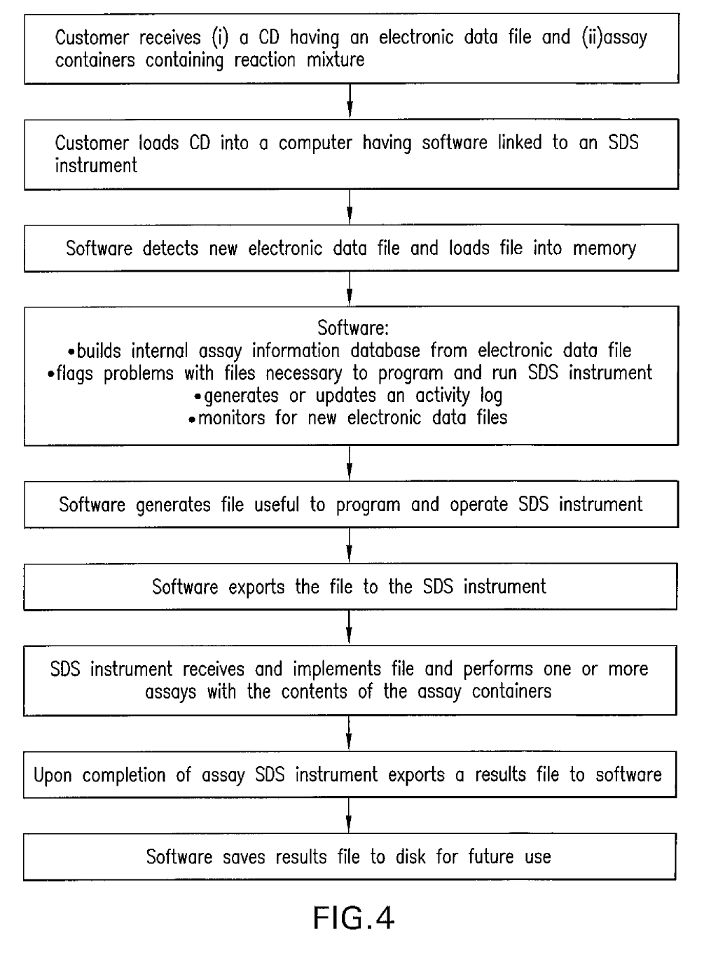

FIG. 4 is a flowchart outlining a method of use of a container and a data storage medium according to an embodiment. A customer can request one or more assays from a supplier. The customer receives from the supplier a CD containing an electronic data file and an assay container or containers, and loads the CD into a CD reader of a computer (e.g., a microcomputer) having software that can communicate with an SDS instrument, for example, an ABI 7900HT system. The software on the microcomputer detects the new electronic data file on the CD and loads the electronic data file into memory. The software can read the contents of the electronic data file and can: build an internal assay information database from the electronic data file; flag problems with any file necessary to run or operate the SDS instrument; generate or update an activity log; and monitor for any new electronic data files. The software can then generate a file or files necessary to program and operate the SDS instrument. The software can then export the file to the SDS instrument. Alternately or additionally, the software can output a printed copy of the protocols and/or parameters used to program the SDS instrument. The SDS instrument can then receive and implement the file and perform the assay or assays using the contents of the assay containers. Upon completion of the assay or assays, the SDS instrument can export to a results file containing an electronic copy of data generated by the assay or assays to the software. The software can then save the results file to a disk, such as, for example, a hard drive or CD, for future use. If desired, the customer can paste the printed copy of the protocols and/or parameters in a lab notebook, for example, or can use the information on the printed copy to manually program the SDS instrument.

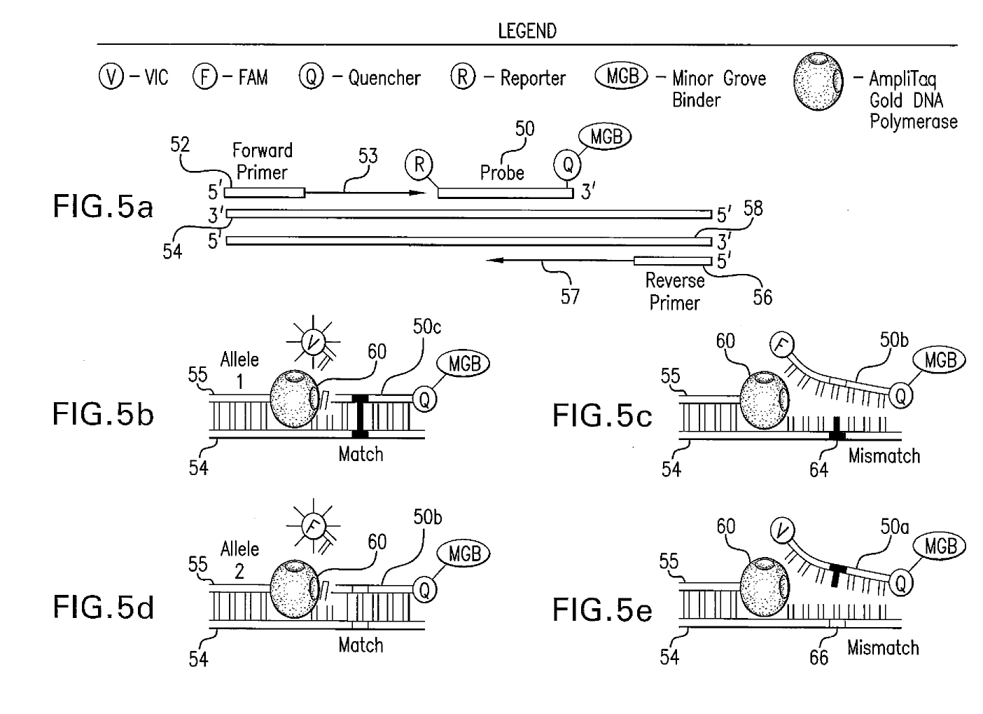

FIG. 5a-5e are schematic diagrams showing the interaction of components that can comprise, at least in part, a homogeneous reaction mixture, according to various embodiments. In FIG. 5a, primer 52 has annealed to template strand 54. Replication of the template strand from primer 52 will occur in the 5′ to 3′ direction. Probe 50, including a generic reporter dye R, quencher Q, and minor groove binder MGB, has annealed to the template strand 54. Arrow 53 shows that as the complementary strand (not shown) is produced from the template strand 54 starting at the forward primer 52, the complementary strand will meet probe 50. FIG. 5b shows the complementary strand 55 as it meets probe 50a. Polymerase 60 cleaves VIC reporter dye V during the production of complementary strand 55 given that probe 50a has annealed to the target strand 54 because the target strand 54 and the probe 50a are completely complementary. FIG. 5c shows the complementary strand 55 as it meets probe 50b. Polymerase 60 does not cleave FAM reporter dye F during the production of complementary strand 55 given that probe 50b has not hybridized with the target strand 54 because of a mismatched base pair at location 64. FIG. 5d shows the complementary strand 55 as it meets probe 50b. Polymerase 60 cleaves FAM reporter dye F during the production of complementary strand 55 given that probe 50b has annealed to the target strand 54 because the target strand 54 and the probe 50b are completely complementary. FIG. 5e shows the complementary strand 55 as it meets probe 50a. Polymerase 60 does not cleave VIC reporter dye V during the production of complementary strand 55 given that the probe 50a has not hybridized with the target strand 54 because of a mismatched base pair at location 66.

Those skilled in the art can appreciate from the foregoing description that the broad teachings herein can be implemented in a variety of forms. Therefore, while the present teachings have been described in connection with various embodiments and examples, the scope of the present teachings should not be so limited.

What is claimed is:

1. A method for facilitating genetic analysis, comprising:
providing a user with software adapted to run on an operating system that controls operation of a sequence detection system in performing an assay;
receiving from the user a request for one or more genetic-analysis assays;
providing to the user (i) one or more homogeneous assays, each homogeneous assay comprising a container and a reaction mixture in the container, the reaction mixture comprising at least one primer and at least one probe, the at least one primer comprising a sequence that is complementary to a target polynucleotide and the at least one probe being reactive with the same target polynucleotide in an allelic discrimination or expression analysis assay when admixed with the target polynucleotide, (ii) a plate rack defining a plurality of locations, wherein each container of the one or more homogeneous assays is disposed in a respective one of the plurality of locations, and (iii) information relating to the one or more homogeneous assays, the information being in electronic form and comprising an assay information file, the assay information file comprising position validation information identifying a predetermined one or more of the plurality of locations defined by the plate rack in which each respective container should be disposed to correspond with the predetermined one or more of the plurality of locations thereof, wherein the position validation information is formatted to be used by the software to control operation of the sequence detection system in performing the one or more homogeneous assays using the reaction mixture of each of the one or more homogeneous assays;
determining whether each respective container is in the respective plate rack location identified by the position validation information; and
controlling operation of the sequence detection system based on what is determined in the determining step.

2. The method of claim 1, wherein the one or more homogeneous assays comprise components for conducting a fluorogenic 5′ nuclease assay.

3. The method of claim 1, wherein the information is provided on a disk.

4. The method of claim 3, wherein the disk is a compact disk (CD).

5. The method of claim 1, wherein the information is provided via a computer network.

6. The method of claim 5, wherein the information is transmitted to the user via electronic mail.

7. The method of claim 5, wherein the information is provided via a file transfer protocol (FTP).

8. The method of claim 1, wherein the information is comprised, at least in part, of American Standard Code for Information Interchange (ASCII) formatted text.

9. The method of claim 1, wherein at least one of the one or more assays and the information are shipped to the user together in a single package.

10. The method of claim 1, wherein the information comprises information about an exon-exon junction.

11. The method of claim 1, wherein the information comprises information about the at least one probe and the at least one primer.

12. The method of claim 1, wherein the information comprises information about one or more nucleic acid sequences to be used in a homogeneous assay.

13. The method of claim 1, wherein the information comprises information about a marker dye.

14. The method of claim 1, wherein the one or more homogeneous assays comprise a homogeneous ligand binding assay.

15. The method of claim 1, wherein a component in the one or more homogeneous assays comprises a minor groove binding probe.

16. The method of claim 1, wherein a component in the one or more homogeneous assays comprises a non-fluorescent quencher.

17. The method of claim 16, wherein the non-fluorescent quencher is at the 3′ end of a polynucleotide.

18. The method of claim 1, wherein a component in the one or more homogeneous assays comprises two target primers and a probe.

19. The method of claim 1, wherein the single-tube format is suitable for use in a sequence detection system instrument.

20. The method of claim 1, wherein the information in electronic form comprises an electronic assay workbook.

21. The method of claim 1, wherein the information comprises at least one of an electronically readable or printable copy of instructions for an assay protocol, an electronically readable or printable copy of protocols for submitting requests for assays, or an electronically readable copy of a product insert.

22. A method for facilitating genetic analysis, comprising:
providing a user with software adapted to run on an operating system that controls operation of a sequence detection system in performing an assay;
receiving from a user a request for one or more genetic-analysis assays;
providing to the user (i) the one or more genetic-analysis assays, each of the one or more genetic-analysis assays provided in a single-tube format, wherein the single-tube format is suitable for use in a fluorescence detection instrument and comprises a container and a reaction mixture in the container, the reaction mixture comprising at least one primer and at least one probe, the at least one primer comprising a sequence that is complementary to a target polynucleotide and the at least one probe being reactive with the same target polynucleotide when admixed with the target polynucleotide, (ii) a plate rack defining a plurality of locations, wherein each container of the one more genetic-analysis assays is disposed in a respective one of the plurality of locations, and (iii) information relating to the one or more genetic-analysis assays, the information being in electronic form and comprising an assay information file, the assay information file comprising position validation information identifying a predetermined one or more of the plurality of locations defined by the plate rack in which each respective container should be disposed to correspond with the predetermined one or more of the plurality of locations thereof, wherein the position validation information is formatted to be used by the software to control operation of the sequence detection system in performing the one or more genetic-analysis assays using the reaction mixture of each of the one or more genetic-analysis assays;
determining whether each respective container is in the respective plate rack location identified by the position validation information; and
controlling operation of the sequence detection system based on what is determined in the determining step.

23. The method of claim 19, wherein the sequence detection system instrument is a fluorescence detection instrument.

24. A method for facilitating genetic analysis comprising:
providing a user with software adapted to run on an operating system that controls operation of a sequence detection system in performing an assay;
receiving from a user a request for one or more genetic-analysis assays;
providing to the user (i) the one or more genetic-analysis assays, each assay being provided in a single-tube format and comprising a container and a reaction mixture in the container, the reaction mixture comprising at least two target primers and at least one probe, the at least two target primers each comprising a sequence that is complementary to a target polynucleotide and the at least one probe being reactive with the same target polynucleotide when admixed with the target polynucleotide, (ii) a plate rack defining a plurality of locations, wherein each container of the one or more genetic-analysis assays is disposed in a respective one of the plurality of locations, and (iii) information relating to the one or more genetic-analysis assays, the information being in electronic form and comprising an assay information file, the assay information file comprising position validation information identifying a predetermined one or more of the plurality of locations defined by the plate rack in which each respective container should be disposed to correspond with the predetermined one or more of the plurality of locations thereof, wherein the position validation information is formatted to be used by the software to control operation of the sequence detection system in performing the one or more genetic-analysis assays using the reaction mixture of each of the one or more genetic-analysis assays;
determining whether each respective container is in the respective plate rack location identified by the position validation information; and
controlling operation of the sequence detection system based on what is determined in the determining step.

25. The method of claim 24, wherein the assay comprises a minor groove binding probe.

26. The method of claim 25, wherein the minor groove binding probe comprises a quencher.

27. The method of claim 24, wherein each assay comprises a reaction mixture for a homogeneous assay.

28. The method of claim 1, further comprising controlling a sequence detection system based on the assay information file to perform an assay using the reaction mixture.

29. The method of claim 23, further comprising controlling a sequence detection system based on the assay information file to perform an assay using the reaction mixture.

30. The method of claim 24, further comprising controlling a sequence detection system based on the assay information file to perform an assay using the reaction mixture.

31. A method for facilitating genetic analysis comprising:
providing a user with software adapted to run on an operating system that controls operation of a sequence detection system in performing an assay;
receiving from a user a request for one or more genetic-analysis assays;
providing to the user (i) the one or more genetic-analysis assays, each assay being provided in a single-tube format and comprising a container and a reaction mixture in the container, the reaction mixture comprising at least two target primers and at least one probe, the at least two target primers each comprising a sequence that is complementary to a target polynucleotide and the at least one probe comprising a dye and being reactive with the same target polynucleotide when admixed with the target polynucleotide, and (ii) information relating to the one or more genetic-analysis assays, the information being in electronic form and comprising an assay information file, the assay information file comprising dye information identifying the dye of the probe, the dye information being formatted to be used by the software to control operation of the sequence detection system in performing the one or more genetic-analysis assays using the reaction mixture of each of the one or more genetic-analysis assays; and
controlling operation of the sequence detection system based on the dye information.
